(19) United States
(12) Patent Application Publication
(10) Pub. No.: US 2005/0182484 A1
(43) Pub. Date: Aug. 18, 2005

(54) Hybrid grafts

(76) Inventor: Umesh H. Patel, West Lafayette, IN (US)

Correspondence Address: Woodard, Emhardt, Moriarty, McNett & Henry LLP;Bank One Center/Tower, 111 Monument Circle, Suite 3700, Indianapolis, IN, 46204-5137, US

(21) Appl. No.: 10/778,648

(22) Filed: Feb. 12, 2004

(51) Int. Cl. A61F 2/06

(52) U.S. Cl. 623/1.36; 623/1.44; 604/8

(57) ABSTRACT

The present application relates to unique surgical grafts. One embodiment of the present application relates to a surgical graft including a synthetic portion and an ECM portion connected thereto.

### BACKGROUND

The present invention relates generally to surgical grafts. More particularly, one embodiment of the present invention relates to a surgical graft including a synthetic material portion and an extracellular matrix material portion. While the present invention was developed for vascular grafts it may also be applied to other biological grafts including, but not limited to, those relating to bile ducts, hepatic ducts, or pancreatic ducts, bypass grafts, grafts relating to cardiac and thoracic surgeries, and other grafts involved in anastomosis.

Surgical grafts are useful in a multitude of surgical applications, for example, to bypass diseased, damaged, occluded, and/or obstructed blood vessels of the heart, limbs, and other locations throughout the body. A further example is an arterio-venous graft (“A-V graft”) which is useful in connection with hemodialysis. An A-V graft is surgically connected between an artery and a vein to permit blood flow therebetween and provide access to the blood stream for performing hemodialysis.

While there are many prior types of grafts, there remains a need for additional technological development in this area. In furtherance of this need, the present application provides a novel and non-obvious graft.

### SUMMARY

In one form the present invention provides unique surgical grafts including an ECM portion and a synthetic portion.

One form of the present invention contemplates a surgical graft, comprising: a synthetic material body including a fluid flow passageway; and an extracellular matrix material body coupled to the synthetic material body, the extracellular matrix material body including a fluid flow passage and an attachment portion adapted to be coupled to human tissue.

Another form of the present invention contemplates a surgical graft, comprising: a graft body including a lumen adapted to permit biological fluid flow therein; and a substantially acellular ECM material connector attached to the graft body and including a second lumen disposed in fluid communication with the lumen, the connector is adapted for surgical attachment to human tissue.

Yet another form of the present invention contemplates a blood vessel graft comprising a body having a fluid flow passageway therethrough, the body including a first portion made of synthetic material and a second portion comprising substantially acellular collagenous tissue matrix material attached thereto, wherein further attachment of the second portion to a blood vessel establishes interconnection of the blood vessel graft and the blood vessel to permit blood flow therebetween.

Yet another form of the present invention contemplates a method comprising: providing a tubular graft including a synthetic portion and a first ECM portion; and
connecting the first ECM portion to a first blood vessel effective to permit blood flow between the blood vessel and the implanted tubular graft in a patient.

One object of the present invention is to provide a unique surgical graft.

Related objects and advantages of the present invention will be apparent from the following description.

### BRIEF DESCRIPTION OF THE DRAWINGS

FIG. 1 is an illustrative perspective view of an anastomosis of a human blood vessel and a vascular graft according to one embodiment of the present invention.

FIG. 2 is an illustrative perspective view of an A-V graft according to another embodiment of the present invention.

FIG. 3 is an illustrative perspective view of a bypass graft according to another embodiment of the present invention.

FIG. 4 is an illustrative partial perspective view of a graft according to another embodiment of the present invention.

FIG. 5 is an illustrative perspective view of a graft according to another embodiment of the present invention.

FIG. 6 is an illustrative perspective view of a graft according to another embodiment of the present invention.

FIG. 7 is an illustrative perspective view of a graft according to another embodiment of the present invention.

FIG. 8 is an illustrative cross sectional view of a graft according to another embodiment of the present invention.

FIG. 9 is an illustrative cross sectional view of a graft according to another embodiment of the present invention.

### DETAILED DESCRIPTION

For the purposes of promoting an understanding of the principles of the invention, reference will now be made to the embodiments illustrated in the drawings and specific language will be used to describe the same. Nevertheless, no limitation of the scope of the invention is thereby intended. Such alterations and further modifications in the illustrated embodiments, and such further applications of the principles of the invention as illustrated therein as would occur to one skilled in the art to which the invention relates, are contemplated.

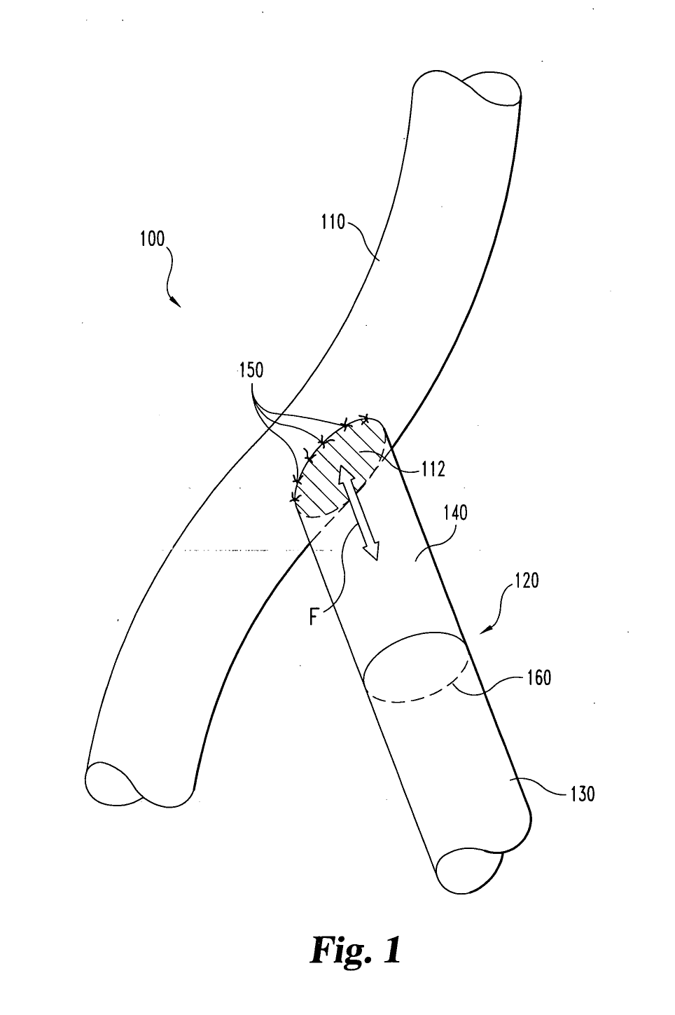

With reference to FIG. 1 there is illustrated an anastomosis 100. In anastomosis 100, blood vessel 110 and vascular graft 120 have been surgically connected with sutures 150. The graft-vessel connection may also be accomplished with staples, glue, stents, clamps, implants or conventional techniques. Opening 112 in blood vessel 110 permits blood flow between the interiors of vessel 110 and graft 120 as generally illustrated by arrow F.

In one form graft 120 includes a non-ECM synthetic material portion 130. The non-ECM synthetic material portion can include synthetic polymeric material such as, but not limited to polytetrafluoroethylene (“PTFE”) (including expanded PTFE) and/or polyethylene terephthalate (“PET). Further, the synthetic polymer materials can be either a biostable or a bioabsorbable polymer. Bioabsorbable polymers that could be used include, but are not limited to, poly(L-lactic acid), polycaprolactone, poly(lactide-co-glycolide), poly(hydroxybutyrate), poly(hydroxybutyrate-co-valerate), polydioxanone, polyorthoester, polyanhydride, poly(glycolic acid), poly(D,L-lactic acid), poly(glycolic acid-co-trimethylene carbonate), polyhydroxyalkanaates, polyphosphoester, polyphosphoester urethane, poly(amino acids), cyanoacrylates, poly(trimethylene carbonate), poly(iminocarbonate), copoly(ether-esters) (e.g., PEO/PLA), polyalkylene oxalates, and polyphosphazenes. Biostable polymers that could be used include, but are not limited to, polyurethanes, silicones, and polyesters and other polymers such as, but not limited to, polyolefins, polyisobutylene and ethylene-alphaolefin copolymers; acrylic polymers and copolymers, vinyl halide polymers and copolymers, such as polyvinyl chloride; polyvinyl ethers, such as polyvinyl methyl ether; polyvinylidene halides, such as polyvinylidene fluoride and polyvinylidene chloride; polyacrylonitrile, polyvinyl ketones; polyvinyl aromatics, such as polystyrene, polyvinyl esters, such as polyvinyl acetate; copolymers of vinyl monomers with each other and olefins, such as ethylene-methyl methacrylate copolymers, acrylonitrile-styrene copolymers, ABS resins, and ethylene-vinyl acetate copolymers; polyamides, such as Nylon 66 and polycaprolactam; alkyd resins, polycarbonates; polyoxymethylenes; polyimides; polyethers; epoxy resins, polyurethanes; rayon; and rayon-triacetate. The material may be in the form of yarns, fibers, and/or resins, monofilament yarns, high tenacity polyester. Further, the present application contemplates other plastic, resin, polymer, woven, and fabric surgical materials, other conventional synthetic surgical materials, and/or combinations of such materials.

Graft 120 also includes an ECM material portion 140. As used herein, ECM material(s) or extracellular matrix materials refer(s) to a class of biomaterials including, but not limited to, submucosa, mucosa, serosa, pericardium, dermis, fascia, basement membrane, and/or combinations thereof. ECM materials may be derived from various tissue sources including the alimentary, hepatic, respiratory, intestinal, integument, urinary, or genital tracts. The portion 140 can include 1, 2, 3, 4, 5, 6, or more ECM layers. ECM materials can be harvested from animals, including, for example, pigs, cattle, sheep or other warm-blooded vertebrates to produce heterologous implants or grafts. Products comprising submucosa tissue derived from porcine small intestine are commercially available ECM material produced by COOK BIOTECH INCORPORATED of West Lafayette, Ind. Portion 140 can comprise any of the aforementioned ECM materials or other ECM materials. Further, in some embodiments, portion 140 can comprise any substantially acellular collagenous matrix, naturally-derived or synthetic. The remainder of the text will refer to the non-synthetic portion as ECM material unless specifically stated to the contrary. This will not, however, be limiting of the broader aspects of the invention.

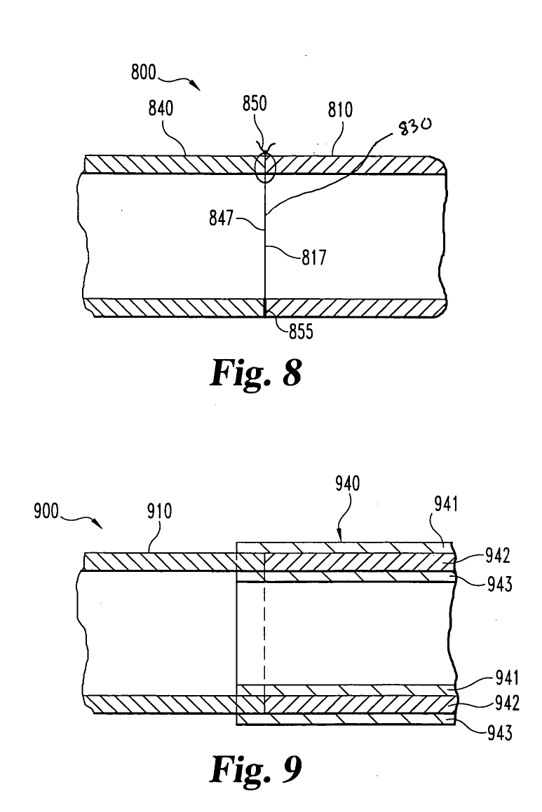

Portions 130 and 140 are connected at connection 160 which may be, for example, of the types described below in connection with FIGS. 8 and 9. Portions 130 and 140 can be of various dimensions for use in various surgical applications. Anastomosis 100 is illustrated as an end-to-side anastomosis, but could also be an end-to-end anastomosis, or any other type as may be appropriate for various surgeries.

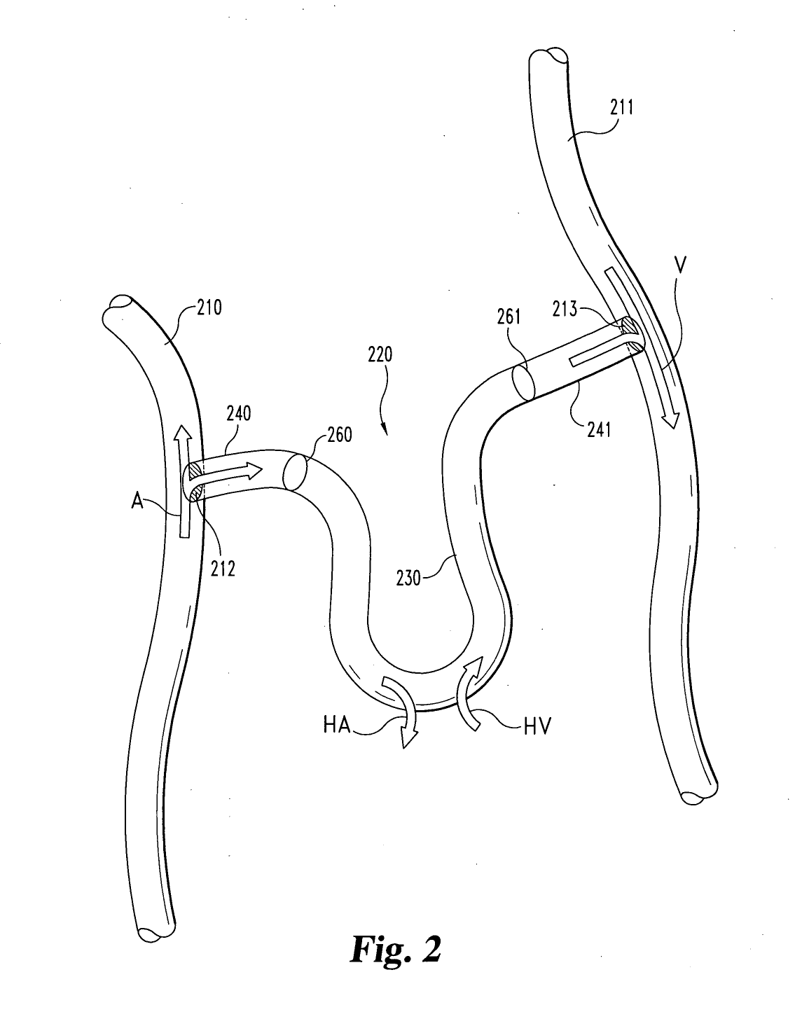

With reference to FIG. 2 there is illustrated one form of an implanted A-V graft 220 useful for hemodialysis. Graft 220 is connected between artery 210 and vein 211. The graft 220 permits blood to flow between the two blood vessels. A portion of the blood flow from artery 210 flows to graft 220 via opening 212 as generally illustrated by arrow A. During hemodialysis, blood is removed from graft 220 as generally shown by arrow HA. This blood is hemodialyzed and returned to graft 220 as generally shown by arrow HV. Blood is then reintroduced to the blood flow of vein 211 via opening 213 as generally shown by arrow V.

A-V graft 220 includes synthetic material portion 230 connected to ECM material portions 240 and 241. The portion 230 being joined to portions 240 and 241 at connections 260 and 261. The graft-vessel connections may be anastomoses as described above in connection with FIG. 1. A-V graft 220 is illustrated as a curved-type graft, but could also be a straight-type or any other type of A-V graft. A-V graft 220 is illustrated as having ECM material portions at both graft-vessel connections, but could also have an ECM material portion at only one end in which case anastomosis at the other end could be between the synthetic portion 230 and a blood vessel or between another portion connected to portion 230 and the blood vessel. In another form the present application further contemplates that the synthetic material portion can be formed by multiple synthetic material pieces connected together by ECM materials and/or other connectors.

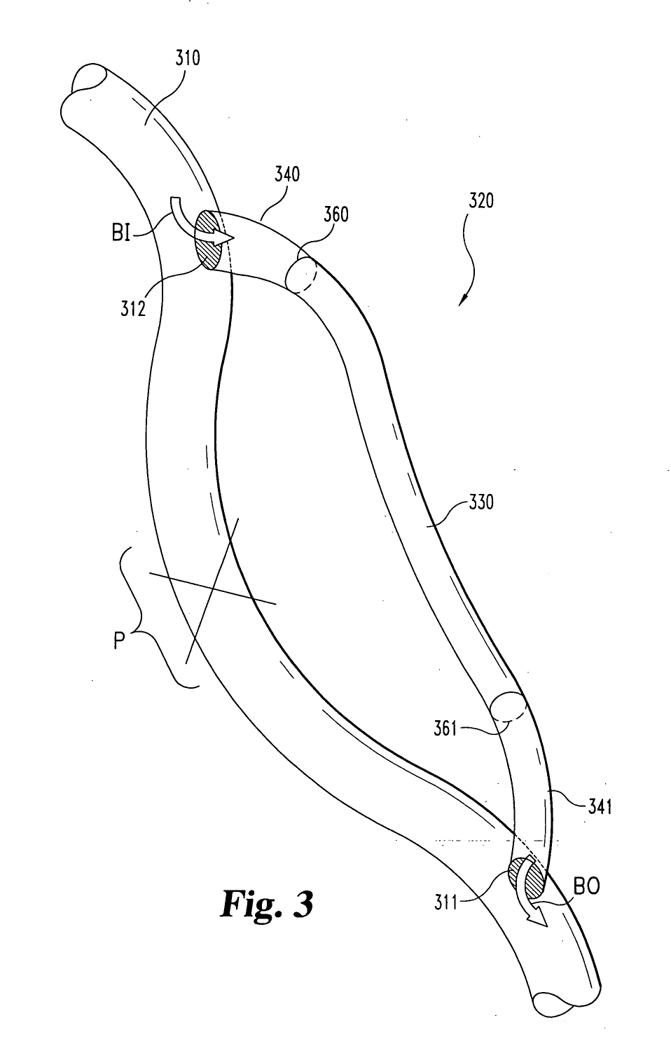

With reference to FIG. 3 there is illustrated an implanted bypass graft 320 useful for bypassing a portion of blood vessel 310, which is generally indicated by X-ed out portion P. The bypassed portion of the vessel is typically damaged or diseased. Graft 320 permits blood flow to bypass portion P as generally illustrated by arrows BI and BO. In one form bypass graft 320 includes synthetic material portion 330, ECM material portions 340 and 341, connections 360 and 361, and openings 311 and 312 which may be, for example, similar to those described above in connection with FIG. 1. The graft-vessel connections may be anastomoses as described above in connection with FIG. 1. A-V graft 320 is illustrated as having ECM material portions at both ends, but could also have an ECM material portion at only one end in which case anastomosis at the other end could be between the synthetic portion 330 and a blood vessel or between another portion connected to portion 330 and the blood vessel. In another form the present application further contemplates that the synthetic material portion can be formed by multiple synthetic material pieces connected together by ECM materials and/or other connectors.

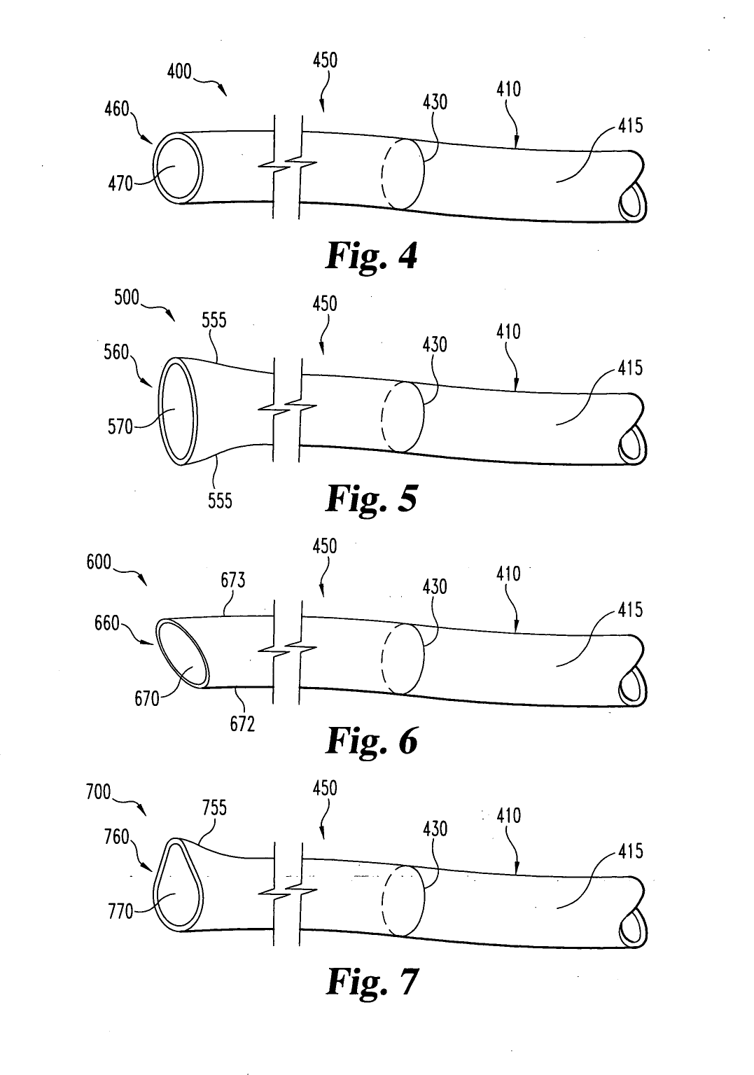

With reference to FIGS. 4-7 there are illustrated a variety of non-limiting examples of vascular grafts according to the present invention. These grafts include similar features indicated with identical reference numerals, but described only once to avoid repetition.

With reference to FIG. 4 there is illustrated vascular graft 400 which includes synthetic material portion 410 and ECM material portion 450 connected at connection 430. Portion 410 may include any of the synthetic materials mentioned above or conventional synthetic materials. Portion 450 may include, for example, any of the materials discussed above in connection with FIG. 1. Connection 430 may be, for example, of the types described below in connection with FIGS. 8 and 9. Graft 400 includes end 460 which defines opening 470. End 460 provides a location for connection to a blood vessel. Opening 470 provides access to the interior 415 of graft 400.

Portion 450 is a single layer ECM material, but can also be a multiple-layer ECM material including, for example, two, three, four, and even more layers of ECM material. Furthermore, portions 410 and 450 may have various lengths, interior and exterior diameters, shapes, curves, bends, thickness, tapers, lumens, flow pathways, junctions, branches, and/or other dimensions and characteristics as may be indicated for a variety of surgical applications.

With reference to FIG. 5 there is illustrated vascular graft 500 which includes numerous features similar to graft 400 indicated with identical reference numerals. Graft 500 includes end 560 which defines opening 570. As shown in FIG. 5, end 560 is partially flattened, opening 570 has a substantially ellipsoid shape, and flares 555 extend outward at end 560. Graft 500 and similar grafts with ends flattened to various degrees permit connection to blood vessels having openings of various shapes.

With reference to FIG. 6 there is shown vascular graft 600. Graft 600 includes many features similar to graft 400 indicated with identical reference numerals. Graft 600 includes end 660 defining opening 670. Opening 670 is formed at an angle with respect to adjacent wall portions 672 and 673. Opening 670 has an ellipsoid shape. Graft 600 and similar grafts with ends formed at various angles permit connection to blood vessels at a variety of angles.

With reference to FIG. 7 there is shown vascular graft 700. Graft 700 includes many similar features to those of graft 400 indicated with identical reference numerals. Graft 700 includes end 760 defining opening 770. End 760 is partially flattened, opening 770 has a tear-drop shape, and flare 755 extends outward at end 760. Graft 700 and similar grafts with ends partially flattened to various degrees permit connection to blood vessels having openings of various shapes.

With reference to FIG. 8 there is shown an illustrative cross sectional view of a vascular graft 800. Graft 800 includes synthetic material portion 810 and ECM material portion 840 which are connected together at connection 830. End 847 of portion 840 and end 817 of portion 810 contact one another. Suture 850 and other sutures may be used to join portions 810 and 840. Instead or in addition, surgical glue 855 or another bonding agent may be used to join portions 810 and 840. Numerous variations on the connection illustrated in FIG. 8 are contemplated. For example, portions 810 and 840 might overlap, portion 810 might extend around or within portion 840 or vice versa, an external and/or internal sleeve might be utilized to connect the portions together, portions 840 might be connected about only a portion of the circumference of connection 830 or vice versa. Furthermore, connection 830 might have a variety of different shapes. For example, connection 830 may be angled, beveled, notched, jagged, curved, and/or tapered, with respect to portion 810 and/or 840 to name a few possibilities.

With reference to FIG. 9 there is shown an illustrative cross sectional view of vascular graft 900 including ECM material portion 940 and synthetic material portion 910. The illustrated ECM material portion includes layers 941 and 943; however ECM material of a differing number of layers is contemplated herein. Synthetic material portion 910 is inserted between layers 941 and 943. Portions 910 and 940 can be joined with one or more sutures and/or surgical glue, for example, as described above in connection with FIG. 8. As set forth above portion 940 is illustrated as including two layers, but could also include fewer or more layers. For example, one or more additional layers might be present adjacent to layer 941 and/or layer 943. In addition, portion 910 which is illustrated as having a uniform thickness could be tapered or stepped to provide a non-uniform thickness for insertion into portion 940.

The present invention contemplates that the ECM material portion can be connected to the human tissue at its end or at any other location along its body.

While embodiments of the invention have been illustrated and described in detail in the drawings and foregoing description, the same is to be considered as illustrative and not restrictive in character, and all changes and modifications that come within the spirit of the invention are desired to be protected. It should be understood that while the use of the word preferable, preferably or preferred in the description above indicates that the feature so described may be more desirable, it nonetheless may not be necessary and embodiments lacking the same may be contemplated as within the scope of the invention, that scope being defined by the claims that follow. In reading the claims it is intended that when words such as “a,” “an,” “at least one,” “a portion,” “at least a portion” are used there is no intention to limit the claim to only one item unless specifically stated to the contrary in the claim. Further, when the language “at least a portion” and/or “a portion” is used the item may include a portion and/or the entire item unless specifically stated to the contrary.

1. A surgical graft, comprising:
a synthetic material body including a fluid flow passageway; and
an extracellular matrix material body coupled to said synthetic material body, said extracellular matrix material body including a fluid flow passage and an attachment portion for coupling to human tissue.

2. The graft of claim 1, wherein said extracellular matrix material body has a first end coupled to said synthetic material body, and wherein said attachment portion is at a second end of said extracellular matrix material body.

3. The graft of claim 2, wherein said fluid flow passageway and said fluid flow passage are disposed in fluid flow communication, and wherein said attachment portion includes a fluid entrance in fluid flow communication with said fluid flow passageway and said fluid passage.

4. The graft of claim 1, wherein said synthetic material body is substantially tubular, and wherein said synthetic material body has a first end and a second end, and further wherein said extracellular matrix material body is coupled to said first end of the synthetic material body.

5. The graft of claim 4, wherein said bodies are fixedly coupled together.

6. The graft of claim 4, wherein said synthetic material body includes PTFE.

7. The graft of claim 1, wherein said extracellular matrix material body includes submucosa.

8. The graft of claim 1, wherein the extracellular matrix material includes a plurality of layers.

9. The graft of claim 8, wherein at least a portion of said synthetic material body is located between a first layer and a second layer of said plurality of layers.

10. The graft of claim 8, wherein at least a portion of said synthetic material body is sandwiched between the layers of said extracellular matrix material body.

11. The graft of claim 1, wherein said extracellular matrix material body and said synthetic material body are coupled together at least in part by glue.

12. The graft of claim 1, wherein said extracellular matrix material body and said synthetic material body are coupled together at least in part by suture.

13. The graft of claim 1, wherein said synthetic material body is substantially tubular, and wherein said synthetic material body has a first synthetic material body end and a second synthetic material body end;
wherein said extracellular matrix material body has a first extracellular matrix material body end coupled to said first synthetic material body end, and wherein said attachment portion is defined at a second extracellular matrix material body end of the extracellular matrix material body;
wherein said fluid flow passageway and said fluid flow passage are in fluid communication, and wherein said attachment portion includes an outlet in fluid flow communication with said fluid flow passageway and said fluid passage; and
wherein said extracellular matrix material body includes submucosa.

14. The graft of claim 1, wherein said synthetic material body is substantially tubular, and wherein said synthetic material body has a first end and a second end;
wherein said extracellular matrix material body is coupled to said synthetic material body at said first end; and
which further includes a second extracellular matrix material body coupled to said synthetic material body at said second end, said second extracellular matrix material body including a second fluid flow passage and a second attachment portion adapted to be coupled to human tissue or organ.

15. The graft of claim 1, wherein said extracellular matrix material body has a first end coupled to said synthetic material body, and wherein said attachment portion is located at a region of said extracellular matrix material body between said first end and a second end of said extracellular matrix material body.

16. The graft of claim 15, wherein said attachment portion includes a fluid entrance in fluid flow communication with said fluid flow passageway and said fluid passage; and
wherein said synthetic material body is substantially tubular and has a first end coupled to said first end of the extracellular matrix material body.

17. A surgical graft, comprising:
a graft body including a lumen adapted to permit biological fluid flow therein; and
a substantially acellular ECM material connector attached to said graft body and including a second lumen disposed in fluid communication with said lumen, said connector adapted for surgical attachment to human tissue.

18. The graft of claim 17, wherein said graft body is formed of synthetic material.

19. The graft of claim 17, wherein said graft body includes a synthetic polymer material.

20. The graft of claim 19, wherein the synthetic material includes one of PET and PTFE.

21. The graft of claim 17, wherein said connector includes a fluid outlet in fluid flow communication with said second lumen.

22. The graft of claim 17, wherein the connector comprises at least a tubular portion.

23. The graft of claim 17, wherein the ECM material consists essentially of acellular, collagen-containing animal tissue.

24. The graft of claim 17, wherein said connector comprises a tubular portion and further includes an outlet in fluid flow communication with said second lumen; and
wherein said graft body is formed of synthetic material, and wherein said graft body is elongated.

25. The graft of claim 17, wherein said graft body includes a first end and a second end, said lumen extending between said first end and said second end;
wherein said connector is attached at said first end; and
which further includes a second substantially acellular ECM material connector adapted for surgical attachment to human tissue, said second connector attached to said graft body at said second end and including a third lumen disposed in fluid communication with said lumen and said second lumen.

26. The graft of claim 17, wherein the graft is a vascular graft.

27. The graft of claim 26, wherein the graft is an A-V graft.

28. A blood vessel graft comprising a body having a fluid flow passageway therethrough, said body including a first portion made of synthetic material and a second portion comprising substantially acellular collagenous tissue matrix material attached thereto, wherein further attachment of the second portion to a blood vessel establishes interconnection of the blood vessel graft and the blood vessel to permit blood flow therebetween.

29. The graft of claim 28, wherein said second portion comprises submucosal tissue.

30. The graft of claim 28, wherein said second portion consists essentially of submucosal tissue.

31. A method comprising:
providing a tubular graft including a synthetic portion and a first ECM portion; and
connecting the first ECM portion to a first blood vessel of a patient effective to permit blood flow between the blood vessel and the implanted tubular graft.

32. The method of claim 30, further comprising connecting the synthetic portion to a second blood vessel effective to permit blood flow between the implanted tubular graft and the second blood vessel.

33. The method of claim 30, wherein the tubular graft includes a second ECM portion spaced from the first ECM portion, and further comprises connecting the second ECM portion to a second blood vessel effective to permit blood flow between the second blood vessel and the implanted tubular graft.
